(19) United States
(12) Patent Application Publication
(10) Pub. No.: US 2026/0347342 A1
(43) Pub. Date: Nov. 5, 2026

(54) AN IMPROVED PROCESS FOR THE PREPARATION OF ERDAFITINIB AND ITS PHARMACEUTICALLY ACCEPTABLE SALTS & POLYMORPHS THEREOF

(71) Applicant: Natco Pharma Limited, Hyderabad (IN)

(72) Inventors: Buchappa Gongalla, Hyderabad (IN); Ravi Babu Kothamasu, Hyderabad (IN); Uma Naresh Babu Kotra, Hyderabad (IN); Kotayya Babu Sikha, Hyderabad (IN); Pulla Reddy Muddasani, Hyderabad (IN); Venkaiah Chowdary Nannapaneni, Hyderabad (IN)

(21) Appl. No.: 19/111,767

(22) PCT Filed: Apr. 18, 2023
(86) PCT No.: PCT/IN2023/050376

§ 371 Date: Mar. 13, 2025

(30) Foreign Application Priority Data
Sep. 14, 2022 (IN) 202241052492

(51) Int. Cl. C07D 403/04

(52) U.S. Cl. CPC C07D 403/04

(57) ABSTRACT

The present invention relates to an improved process for the preparation of N-(3,5dimethoxyphenyl)-N′-(1-methylethyl)-N-[3-(1-methyl-1H-pyrazol-4-yl) quinoxalin-6-yl]ethane-1,2-diamine compound of formula-1 which is represented by the following structural formula.

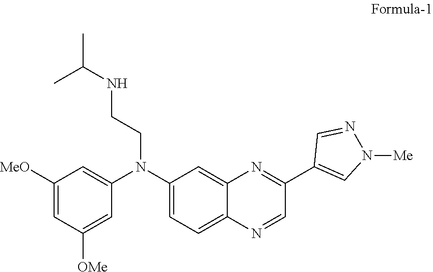 [SMILES: COc1cc(OC)cc(N(CCNC(C)C)c2ccc3ncc(-c4cnn(C)c4)nc3c2)c1]

### FIELD OF THE INVENTION

The present invention relates to an improved process for the preparation of N-(3,5dimethoxyphenyl)-N′-(1-methylethyl)-N-[3-(1-methyl-1H-pyrazol-4-yl) quinoxalin-6-yl]ethane-1,2-diamine compound of formula-1 which is represented by the following structural formula:

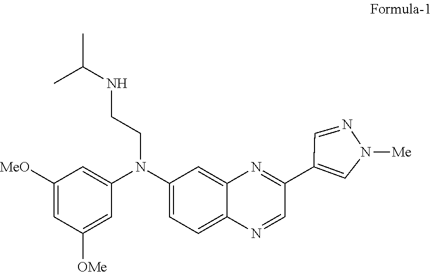 [SMILES: COc1cc(OC)cc(N(CCNC(C)C)c2ccc3ncc(-c4cnn(C)c4)nc3c2)c1]

The present invention also relates to salts of compound of formula-1 and its crystalline forms as well as its process for the preparation.

### BACKGROUND OF THE INVENTION

The drug compound N-(3,5-dimethoxyphenyl)-N′-(1-methylethyl)-N-[3-(1-methyl-1H-pyrazol-4-yl)quinoxalin-6-yl]ethane-1,2-diamine is commonly known as “Erdafitinib”. The Erdafitinib is a fibroblast growth factor receptor (FGFR) kinase inhibitor indicated for the treatment of adult patients with locally advanced or metastatic urothelial carcinoma.

Erdafitinib has been approved by USFDA on 12 Apr. 2019 and sold under the brand name “Balversa”.

U.S. Pat. No. 8,895,601 disclosed a process for the preparation of Erdafitinib. The process described in U.S. Pat. No. 8,895,601 having many drawbacks such as process involves more number of steps, column purifications and provides the final compound with low yield and purity. Hence, the said process is not suitable for commercial scale process.

Therefore, there remains a need to provide a cost effective and commercially viable process for the preparation Erdafitinib.

Polymorphism, the occurrence of different crystal forms, is a property of some molecules and molecular complexes. A single molecule, may give rise to a variety of crystalline forms having distinct crystal structures and physical properties like melting point, X-ray diffraction pattern, infrared absorption fingerprint, and solid state NMR spectrum, thermogravimetric analysis (‘TGA’), and differential scanning calorimetry (‘DSC) which have been used to distinguish polymorphic forms.

As of the date, the PXRD of Erdafitinib HCl was not characterized and disclosed in the prior-art. The present inventors have characterized and disclosed the PXRD of Erdafitinib HCl and designated as crystalline “Form-N”.

### BRIEF DESCRIPTION OF THE INVENTION

The first aspect of the present invention is to provide an improved process for the preparation of Erdafitinib compound of formula-1.

The second aspect of the present invention is to provide a crystalline form of Erdafitinib HCl compound of formula-1a hereinafter designated as “Form-N” and its process for the preparation.

The third aspect of the present invention is to provide an improved process for the preparation of crystalline form-A of Erdafitinib compound of formula-1.

### BRIEF DESCRIPTION OF THE DRAWINGS

FIG. 1: Illustrates the characteristic PXRD pattern of crystalline form-N of Erdafitinib HCl (Formula-1a).

FIG. 2: Illustrates the characteristic PXRD pattern of crystalline form-A of Erdafitinib.

### DETAILED DESCRIPTION OF THE INVENTION

The term “suitable solvent” used in the present invention refers to “hydrocarbon solvents” selected from aliphatic hydrocarbon solvents such as n-hexane, n-heptane, cyclohexane, petroleum ether and aromatic hydrocarbon solvents such as toluene, xylene and the like; “ether solvents” such as dimethyl ether, diisopropyl ether, diethyl ether, methyl tert-butyl ether, 1,2-dimethoxyethane, tetrahydrofuran, 1,4-dioxane, monoxime, dioxime and the like; “ester solvents” such as methyl acetate, ethyl acetate, isopropyl acetate, n-butyl acetate and the like; “polar-aprotic solvents such as N, N-dimethylacetamide, N,N-dimethylformamide, dimethyl sulfoxide, N-methyl pyrrolidone (NMP) and the like; “chloro solvents” such as dichloromethane/methylene chloride, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcoholic solvents” such as methanol, ethanol, n-propanol, isopropanol, n-butanol, isobutanol, t-butanol and the like; “polar solvents” such as water or mixtures thereof.

As used herein the present invention, the term “anti-solvent” refers to a solvent which is used to precipitate the solid from a solution.

As used herein the present invention the term “suitable acid” refers to organic acids or inorganic acids. The “inorganic acid” is selected from hydrochloric acid, hydrobromic acid, sulfuric acid, nitric acid and phosphoric acid; and “organic acid” is selected from formic acid, acetic acid, propionic acid, butyric acid, valeric acid, caproic acid, trifluoroacetic acid, trifluoromethanesulfonic acid, oxalic acid, malonic acid, maleic acid, fumaric acid, malic acid, succinic acid, citric acid, aspartic acid, tartaric acid, mandelic acid, benzoic acid, salicylic acid, substituted/unsubstituted alkyl/aryl sulfonic acids such as methanesulfonic acid, ethanesulfonic acid, propanesulfonic acid, benzenesulfonic acid, p-toluenesulfonic acid, naphthalenesulfonic acid and the like or mixtures thereof.

As used herein the present invention the term “suitable base” refers to “alkali metal carbonates” such as sodium carbonate, potassium carbonate, lithium carbonate, cesium carbonate and the like; “alkali metal bicarbonates” such as sodium bicarbonate, potassium bicarbonate and the like; “alkali metal hydroxides” such as sodium hydroxide, potassium hydroxide, lithium hydroxide and the like; “alkali metal alkoxides” such as sodium methoxide, sodium ethoxide, potassium methoxide, potassium ethoxide, sodium tert-butoxide, potassium tert-butoxide, lithium tert-butoxide and the like; alkali metal hydrides such as sodium hydride, potassium hydride, lithium hydride and the like; alkali metal amides such as sodium amide, potassium amide, lithium amide and the like; Ammonia; and organic bases like dimethylamine, diethylamine, diisopropylamine, diisopropylethylamine, diisobutylamine, triethylamine, pyridine, 4-dimethylaminopyridine (DMAP), N-methyl morpholine (NMM), 2,6-lutidine, lithium diisopropylamide; organosilicon bases such as lithium hexamethyldisilazide (LiHMDS), sodium hexamethyldisilazide (NaHMDS), potassium hexamethyldisilazide (KHMDS) or mixtures thereof.

The first aspect of the present invention is to provide an improved process for the preparation of Erdafitinib compound of formula-1, comprising of:
a) Treating 7-Bromo-1H-quinoxalin-2-one compound of formula-2 with thionyl chloride

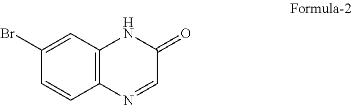 [SMILES: O=c1cnc2ccc(Br)cc2[nH]1]

in presence of N, N-dimethylformamide as a catalyst in absence of solvent to provide 7-Bromo-2-chloroquinoxaline compound of formula-3, optionally purifying the obtained compound with a suitable solvent,

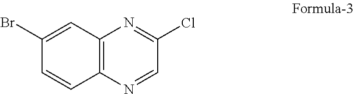 [SMILES: Clc1cnc2ccc(Br)cc2n1]

b) reacting the compound of formula-3 with 1-Methylpyrazole-4-boronic acid pinacol ester compound of formula-4

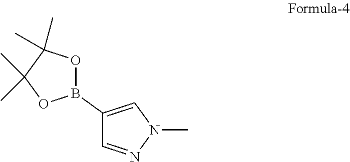

in presence of Sodium carbonate as a base, tetrakis(triphenyl phosphine)palladium [Pd(PPh₃)₄] as catalyst and triphenyl phosphine as ligand in a suitable solvent to provide 7-Bromo-2-(1-methyl-1H-pyrazol-4yl) quinoxaline compound of formula-5, optionally purifying the obtained compound with a suitable solvent,

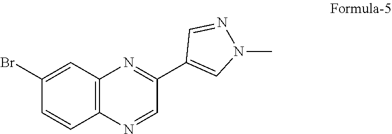 [SMILES: Cn1cc(-c2cnc3ccc(Br)cc3n2)cn1]

c) reacting the compound of formula-5 with 3,5-Dimethoxyaniline compound of formula-6

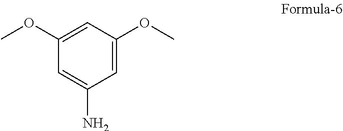

in presence of Cesium carbonate as a base, Palladium(II)acetate as a catalyst, Xantphos as a ligand in a suitable solvent to provide N-(3,5-dimethoxyphenyl)-3-(1-methylpyrazol-4-yl)quinoxaline-6-amine compound of formula-7, optionally purifying the obtained compound with a suitable solvent,

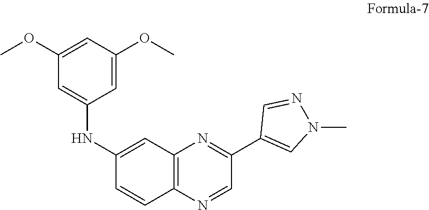

d) reacting the compound of formula-7 with N-(2-chloroethyl)-2-propanamine·HCl compound of formula-8,

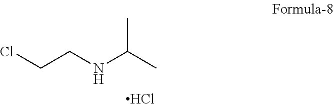

in presence of potassium hydroxide as a base, tetrabutyl ammonium bromide (TBAB) as a catalyst in a suitable solvent to provide N-(3,5-dimethoxyphenyl)-N′-(1-methylethyl)-N-[3-(1-methyl-1H-pyrazol-4-yl) quinoxalin-6-yl]ethane-1,2-diamine crude compound of formula-1, followed by treating the obtained compound with IPA·HCl to provide Erdafitinib HCl salt compound of formula-1a, optionally purifying the obtained compound with a suitable solvent,

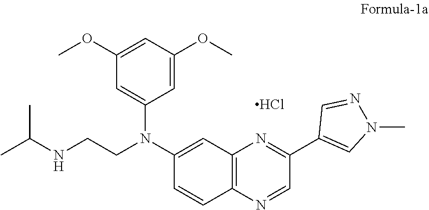 [SMILES: COc1cc(OC)cc(N(CCNC(C)C)c2ccc3ncc(-c4cnn(C)c4)nc3c2)c1 ; Cl]

e) treating the compound of formula-1a with sodium hydroxide in a suitable solvent to provide Erdafitinib compound of formula-1.

Wherein,
in step-a, b, c, d & e) the suitable solvent is selected from alcoholic solvents, polar-aprotic solvents, hydrocarbon solvents, ester solvents, ether solvents, ketone solvents, chloro solvents, nitrile solvents and polar solvents such as water or mixtures thereof.

The preferred embodiment of the present invention is to provide an improved process for the preparation of Erdafitinib compound of formula-1, comprising of:
a) Treating 7-Bromo-1H-quinoxalin-2-one compound of formula-2 with thionyl chloride in presence of N, N-dimethylformamide to provide 7-Bromo-2-chloroquinoxaline compound of formula-3, purifying the obtained compound with 15% ethyl acetate in hexane to obtain compound of formula-3 with high purity and yield,
b) reacting the compound of formula-3 with 1-Methylpyrazole-4-boronic acid pinacol ester compound of formula-4 in presence of Sodium carbonate as a base, tetrakis(triphenyl phosphine)palladium [Pd(PPh₃)₄] as catalyst and triphenyl phosphine as ligand in dimethyl sulfoxide (DMSO) solvent to provide 7-Bromo-2-(1-methyl-1H-pyrazol-4yl)quinoxaline compound of formula-5, purifying the obtained compound with 20% ethyl acetate in hexane to obtain compound of formula-5 with high purity and yield,
c) reacting the compound of formula-5 with 3,5-Dimethoxyaniline compound of formula-6 in presence of Cesium carbonate as a base, Palladium(II)acetate as a catalyst, Xantphos as a ligand in 1,2-dimethoxyethane solvent to provide N-(3,5-dimethoxyphenyl)-3-(1-methylpyrazol-4-yl)quinoxaline-6-amine compound of formula-7, purifying the obtained compound with 25% tetrahydrofuran in ethyl acetate solvent to obtain compound of formula-7 with high purity,
d) reacting the compound of formula-7 with N-(2-chloroethyl)-2-propanamine·HCl compound of formula-8 in presence of potassium hydroxide as a base, tetrabutylammonium bromide (TBAB) as a catalyst in aqueous tetrahydrofuran solvent to provide N-(3,5-dimethoxyphenyl)-N′-(1-methylethyl)-N-[3-(1-methyl-1H-pyrazol-4-yl)quinoxalin-6-yl]ethane-1,2-diamine crude compound of formula-1, followed by treating the obtained compound with IPA·HCl in isopropyl alcohol solvent to provide Erdafitinib HCl salt compound of formula-1a which was purified with acetone and dichloromethane to obtain compound of formula-1a with high purity,
e) treating the compound of formula-1a with sodium hydroxide in ethyl acetate to provide Erdafitinib compound of formula-1, isolating the obtained compound with aqueous acetone to provide the pure Erdafitinib compound of formula-I.

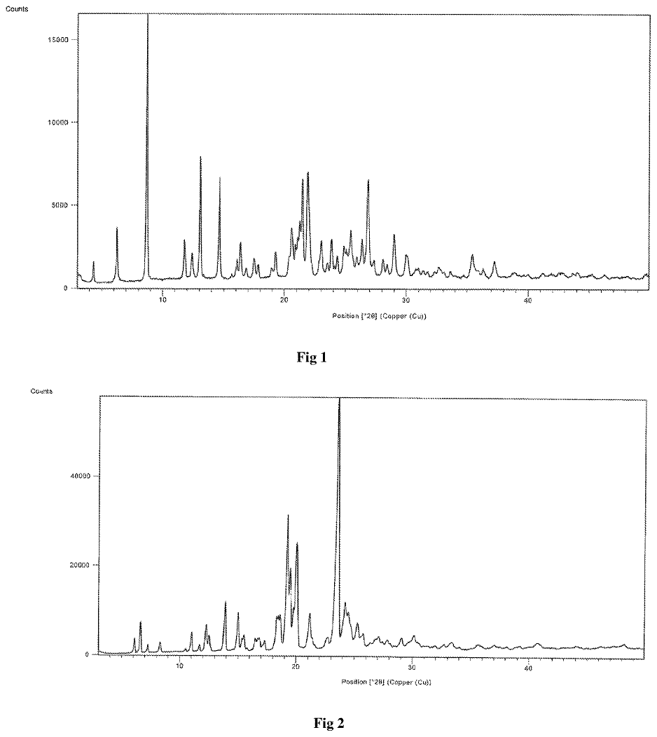

The second aspect of the present invention is to provide a crystalline form-N of Erdafitinib HCl salt compound of formula-1a, which is characterized by:
i) Its powder X-ray diffractogram having peaks at about 6.2, 8.7, 13.0, 14.6, 21.4, 21.8 and 26.8±0.2 degrees 2-theta.
ii) Its powder X-ray diffraction pattern as shown in FIG. 1.

Further, the present invention also provides a process for the preparation of crystalline Form-N of Erdafitinib HCl salt compound of formula-1a, comprising of:
a) Adding isopropyl alcohol to N-(3,5-dimethoxyphenyl)-N′-(1-methylethyl)-N-[3-(1-methyl-1H-pyrazol-4-yl)quinoxalin-6-yl]ethane-1,2-diamine crude compound of formula-1,
b) heating and stirring the reaction mixture,
c) adding IPA·HCl to the reaction mass,
d) cooling and stirring the reaction mixture,
e) adding a suitable solvent to the reaction mass,
f) filtering, washing and drying the compound to get the crystalline form-N of Erdafitinib HCl salt compound of formula-1a.

Wherein in step-a) & e), the suitable solvent is selected from ether solvents, chloro solvents, ester solvents, alcohol solvents, ketone solvents, polar aprotic solvents, hydrocarbon solvents, nitrile solvents and polar solvents or mixtures thereof.

The preferred embodiment of the present invention provides a process for the preparation of crystalline Form-N of Erdafitinib HCl salt compound of formula-1a, comprising of:
a) Adding isopropyl alcohol to N-(3,5-dimethoxyphenyl)-N′-(1-methylethyl)-N-[3-(1-methyl-1H-pyrazol-4-yl)quinoxalin-6-yl]ethane-1,2-diamine crude compound of formula-1,
b) heating and stirring the reaction mixture,
c) adding IPA·HCl to the reaction mass,
d) cooling and stirring the reaction mixture,
e) adding acetone & dichloromethane to the reaction mass,
f) filtering, washing with acetone and drying the compound to get the crystalline form-N of Erdafitinib HCl salt compound of formula-1a.

The third aspect of the present invention is to provide a process for the preparation of crystalline Form-A of Erdafitinib compound of formula-1, comprising of.
a) Adding a suitable solvent to Erdafitinib HCl compound of formula-1a,
b) neutralizing the reaction mass with a suitable base,
c) adding a suitable solvent and heating the reaction mass,
d) stirring and cooling the reaction mixture,
e) filtering, washing the wet solid with a suitable solvent and drying the compound to get the crystalline Form-A of Erdafitinib compound of formula-1.

Wherein in step-b), the suitable base is sodium hydroxide.

In step-a) & c), the suitable solvents are selected from ether solvents, chloro solvents, ester solvents, alcohol solvents, ketone solvents, polar aprotic solvents, hydrocarbon solvents, nitrile solvents and polar solvents such as water or mixtures thereof.

The preferred embodiment of the present invention is to provide a process for the preparation of crystalline Form-A of Erdafitinib compound of formula-1, comprising of:
a) Adding ethyl acetate to Erdafitinib HCl compound of formula-1a,
b) neutralizing the reaction mass with sodium hydroxide,
c) adding aqueous acetone and heating the reaction mass,
d) stirring and cooling the reaction mixture,
e) filtering, washing the wet solid with aqueous acetone and drying the compound to get the crystalline Form-A of Erdafitinib compound of formula-1.

The 7-Bromo-1H-quinoxalin-2-one compound of formula-2 and 1-Methylpyrazole-4-boronic acid pinacol ester compound of formula-4 & 3,5-Dimethoxyaniline compound of formula-6 are prepared from the processes known in the art.

Advantages of the present invention:
1. The compound of formula-3 is prepared in absence of solvent thereby maintenance time reduced from 17 hrs to 2-3 hrs by performing neat reaction.
2. The process of the present invention does not require chromatographic purification techniques like column chromatography, preparative HPLC or preparative TLC in any stage of the process.
3. In the process for preparing compound of formula-7, the maintenance time reduced from 17-20 hrs to 8-10 hrs by using Xantphos as ligand in place of Rac-BiNap.
4. The preparation of HCl salt compound of formula-1a, isolating and purification of salt provides Erdafitinib with high purity and yield.
5. The present invention involves the usage of low cost reagents & solvents which reduce the cost of production and also best suitable for commercial scale process.

PXRD method of analysis:
PXRD analysis of the crystalline form of Erdafitinib and its HCl salt were carried out using Panlytical Expert Pro DY3248 X-ray powder diffractometer using Cu-Ka radiation of 10 wavelength 1.5406 A° and at continuous scan speed of 0.03°/min.

The process for the preparation of Erdafitinib compound of formula-1 is schematically represented as below:

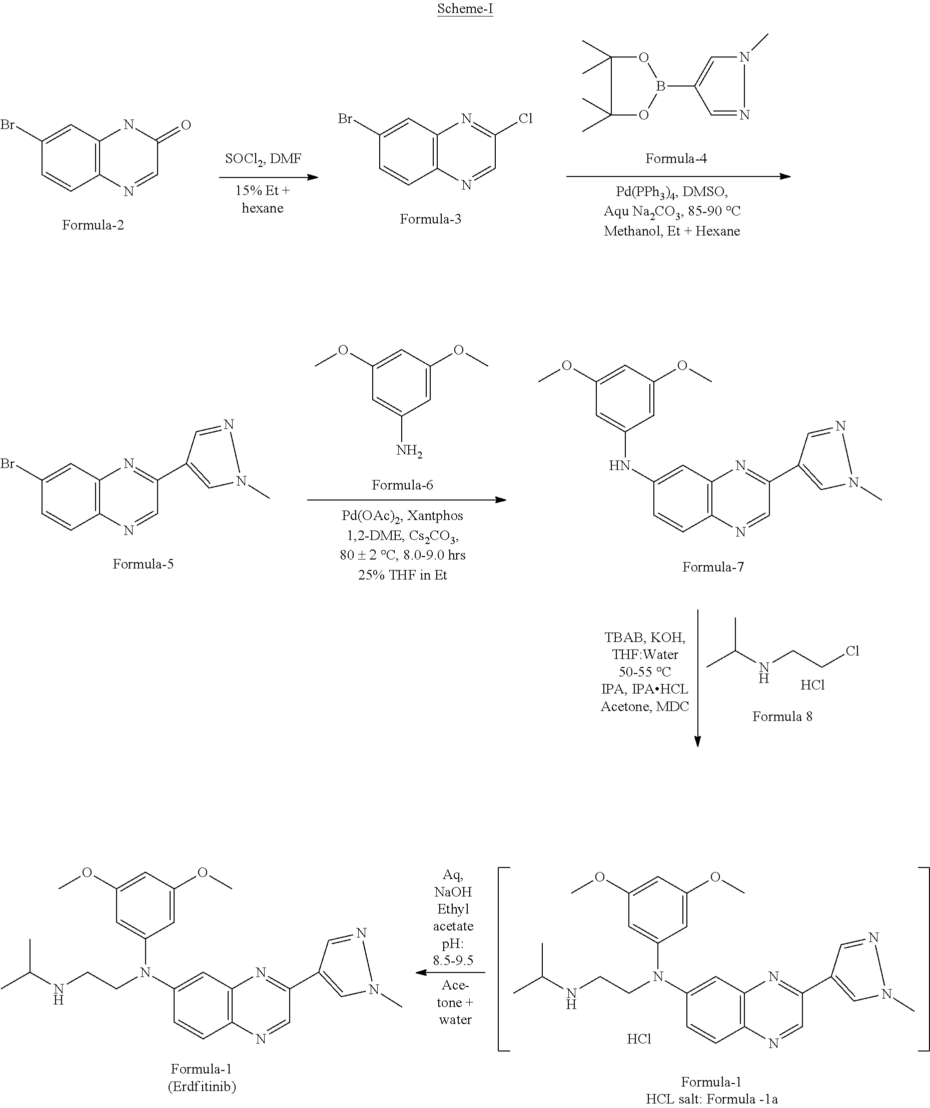

The best mode of carrying out the present invention was illustrated by the below mentioned examples. These examples provide as illustration only and hence should not be construed as limitation of the scope of the invention.

### EXAMPLES

### Example-1: Preparation of 7-Bromo-2-chloroquinoxaline (Formula-3)

Into a 4-neck RBF charged 7-Bromo-1H-quinoxalin-2-one compound of formula-2 (250 g) and thionyl chloride (1500 mL) and DMF (8.12 g). Heated the reaction mass to 70-75 °C and stirred for 2-3 hours at same temperature and then cooled to 25-30 °C. Subsequently, Charged DM Water (7500 mL) into 4-Neck RB flask and cooled to 5 °C and slowly added the obtained thionyl chloride reaction mass and then stirred for 20-30 mins at same temperature. Filtered the compound and transferred into another 4-neck RBF and then charged 2500 mL of DM Water and stirred for 20-30 mins at 25-30 °C and filtered the reaction mass. Charged 15% Ethyl acetate in Hexane (2500 mL) and obtained crude into another 4N RBF and heated to 50-60 °C and then stirred for 45-60 mins at same temperature. Cooled the reaction mass temperature to 25-30 °C and then stirred for 45-60 mins at same temperature. Filtered the reaction mass and washed with 15% Ethyl acetate in hexane (500 mL) and then dried to provide the title compound 7-Bromo-2-chloroquinoxaline (Formula-3) (Yield: 211 gms).

### Example-2: Preparation of 7-Bromo-2-(1-methyl-1H-pyrazol-4yl) quinoxaline (Formula-5)

DM Water (500 mL) and Na₂CO₃ (108.8 g) and DMSO (2400 mL) were charged into 4N RBF and then charged 7-Bromo-2-chloroquinoxaline (Formula-3) (200 g) and 1-Methylpyrazole-4-boronic acid pinacol ester (Formula-4) (188 g, Lot-I) and Triphenylphosphine (8.62 g) into reaction mass and then bubbled the reaction mass with N₂ for 10-15 mins. Charged Pd(PPh₃)₄ (3.8 g) into reaction mass and again bubbled with N₂ for 10-15 mins. Heated the reaction mass temperature to 85-90 °C and stirred for 18 hours at same temperature. Cooled the reaction mass temperature to 25-35 °C and stirred for 90-120 mins at same temperature and then filtered the reaction mass.

Charged DM water (2000 mL) and above obtained solid mass into 4N RBF and stirred for 20-30 mins at 25-35 °C and then filtered. Charged methylene chloride (3000 mL) and obtained crude into another 4N RBF, heated the reaction mass temperature to 35-40 °C and stirred for 20-30 mins at same temperature. Transferred the reaction mass into a separating funnel in hot condition, settled and collected the organic layer and then distilled off. Charged Methanol (1600 mL) and obtained solid into another 4N RBF. Heated the reaction mass temperature to 60-65 °C and stirred for 45-60 mins at same temperature and then cooled to 25-30 °C and stirred for 45-60 mins. Filtered the reaction mass and transferred into another 4N RBF and charged 20% Ethyl acetate in hexane (2000 mL), heated the reaction mass temperature to 50-60 °C and stirred for 45-60 mins. Cooled the reaction mass temperature to 25-30 °C and stirred for 45-60 mins at same temperature. Filtered the reaction mass and washed with 20% Ethyl acetate in hexane (200 mL) and then dried to provide the title compound 7-Bromo-2-(1-methyl-1H-pyrazol-4yl) quinoxaline (Formula-5) (Yield: 163 gms with above 99% purity).

### Example-3: Preparation of N-(3,5-dimethoxyphenyl)-3-(1-methylpyrazol-4-yl) quinoxaline-6-amine (Formula-7)

7-Bromo-2-(1-methyl-1H-pyrazol-4yl) quinoxaline (Formula-5) (150 g), 1,2-Dimethoxyethane (2250 mL) and 3,5-Dimethoxyaniline (Formula-6)(103.30 g) were charged into 4N RBF and charged Cs₂CO₃ (202.80 g) and Xantphos (5.4 g) into reaction mass and then bubbled the reaction mass with N₂ for 10-15 mins. Charged Pd(OAc)₂ (1.05 g) into reaction mass and again bubbled with N₂ for 10-15 mins. Heated the reaction mass temperature to 78-82 °C and stirred for 8-9 hours at same temperature.

Cooled the reaction mass temperature to 50 °C and charged DM Water (4500 mL) and then cooled to 25-30 °C. Stirred for 45-60 mins at same temperature and then filtered the reaction mass.

Charged Ethyl acetate (3000 mL) and above obtained wet solid mass into 4N RBF and heated to 63-67 °C and then stirred the reaction mass at same temperature for 20-30 mins. Transferred the reaction mass into separating funnel in hot condition and collected the organic layer, filtered, and then distilled off completely. Charged 25% THE in Ethyl acetate (1500 mL) and obtained crude was transferred into another 4N RBF, heated the reaction mass temperature to 60-65 °C and stirred for 45-60 mins at same temperature. Cooled the reaction mass temperature to 25-30 °C and stirred for 45-60 mins. Filtered the reaction mass and washed with 25% THE in Ethyl acetate and then dried to provide the title compound N-(3,5-dimethoxyphenyl)-3-(1-methylpyrazol-4-yl)quinoxaline-6-amine (Formula-7) (Yield: 139 gms with above 99% purity).

### Example-4: Preparation of N-(3,5-dimethoxyphenyl)-N′-(1-methylethyl)-N-[3-(1-methyl-1H-pyrazol-4-yl)quinoxalin-6-yl]ethane-1,2-diamine crude (Formula-1)

Into a 4N RBF charged KOH (388 g), DM water (62.5 mL), THE (3125 mL), N-(3,5-dimethoxyphenyl)-3-(1-methylpyrazol-4-yl)quinoxaline-6-amine (Formula-7)(125 g) and Tetrabutylammonium bromide (TBAB) (27.86 g). Heated the reaction mass temperature to 50-55 °C and stirred for 60-75 mins. Charged N-(2-chloroethyl)-2-propanamine·HCl (Formula-8) (120.3 g) and stirred for 12-13 hours at same temperature. Cooled the reaction mass temperature to 25-35 °C and charged DM water (1250 mL) and stirred for 20-30 mins and then transferred the reaction mass into a separating funnel and collected organic layer. Charged organic layer into another 4N RBF and charged Carbon (12.5 g) and then stirred for 20-30 mins. Filtered the reaction mass on a hyflo bed and distilled off solvent from filtrate to provide the title compound N-(3,5dimethoxyphenyl)-N′-(1-methylethyl)-N-[3-(1-methyl-1H-pyrazol-4-yl) quinoxaline-6-yl]ethane-1,2-diamine crude (Formula-1 crude). (Yield: 168 gms; Purity: 96% by HPLC)

### Example-5: Preparation of Erdafitinib HCl Salt Compound of Formula-1a

Charged IPA (100 mL) and above obtained crude (Formula-1) into 4N RBF and heated the reaction mass temperature to 60-65 °C and then stirred for 10-15 mins. Added IPA. HCl (191.3 g) to the reaction mass at 60-65 °C and stirred for 30-45 mins at same temperature. Cooled the reaction mass to 25-35 °C and stirred for 45-60 mins and then filtered and washed with acetone (500 mL). Charged obtained HCl salt and Acetone (1875 mL) into 4NRBF and heated the reaction mass temperature to 50-55 °C and then stirred for 30-45 mins. Cooled the reaction mass temperature to 25-35 °C and stirred for 30-45 mins and then filtered and washed with acetone (500 mL). Charged obtained wet material and Methylene chloride (750 mL) into 4N RBF and stirred the reaction mass at 25-35 °C for 30-45 mins and then filtered and washed with acetone (500 mL). (Yield: 210 gms; Purity: 99.7% by HPLC)

The PXRD of Erdafitinib HCl salt compound of formula-1a is illustrated in FIG. 1.

### Example-6: Preparation of Erdafitinib Crystalline Form-A

Charged above obtained compound of Formula-1a wet material to DM water in 4N RBF and charged Ethyl acetate (1875 mL). Adjusted the reaction mass pH to 8.5-9.5 by using aq·NaOH solution stirred the reaction mass for 10-15 mins. Transferred the reaction mass into a separating funnel, settled and separated the organic layer (lot-I) and aqueous layer. Charged the collected aqueous layer into 4N RBF and charged ethyl acetate (625 mL) and stirred for 10-15 mins. Transferred the reaction mass into a separating funnel, settled and separated the organic layer (lot-II). Combine two lots of organic layers and transferred into 4N RBF and charged DM water (625 mL) and then stirred for 10-15 mins. Separated the organic layer and distilled off completely.

Charged Acetone (1000 mL) into the above Erdafitinib, heated to 50-55 °C and stirred for 10-15 mins at same temperature. Added DM water (2500 mL) into reaction mass over a period of 45-60 mins at 50-55 °C and stirred for 20-30 mins at same temperature. Cooled the reaction mass to 25-35 and stirred for 3-4 hours at same temperature. Filtered the reaction mass and washed with DM water (500 mL) and then dried to provide the title compound Erdafitinib crystalline form-A (Yield: 114 gms; Purity: 99.8% by HPLC).

The PXRD of Erdafitinib compound of formula-1 is illustrated in FIG. 2.

We claim:

1. An improved process for the preparation of Erdafitinib compound of formula-1, 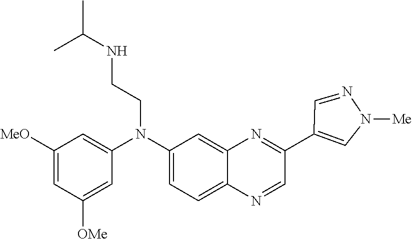 [SMILES: COc1cc(OC)cc(N(CCNC(C)C)c2ccc3ncc(-c4cnn(C)c4)nc3c2)c1]
comprising of:
a) Treating 7-Bromo-1H-quinoxalin-2-one (Formula-2) with thionyl chloride 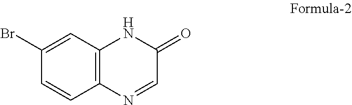 [SMILES: O=c1cnc2ccc(Br)cc2[nH]1]
in presence of N, N-dimethylformamide as a catalyst in absence of a solvent to provide 7-Bromo-2-chloroquinoxaline (Formula-3), optionally purifying the obtained compound with a suitable solvent, 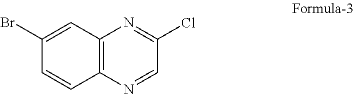 [SMILES: Clc1cnc2ccc(Br)cc2n1]
b) reacting the compound of formula-3 with 1-Methylpyrazole-4-boronic acid pinacol ester (Formula-4) 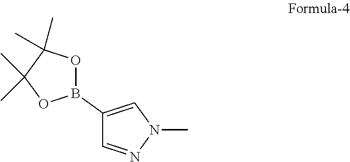
in presence of Sodium carbonate as a base, tetrakis(triphenyl phosphine)palladium [Pd(PPh₃)₄] as catalyst and triphenyl phosphine as ligand in a suitable solvent to provide 7-Bromo-2-(1-methyl-1H-pyrazol-4yl) quinoxaline (Formula-5), optionally purifying the obtained compound with a suitable solvent, 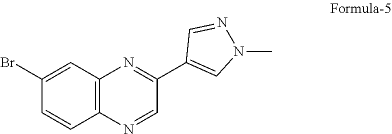 [SMILES: Cn1cc(-c2cnc3ccc(Br)cc3n2)cn1]
c) reacting the compound of formula-5 with 3,5-Dimethoxyaniline (Formula-6) 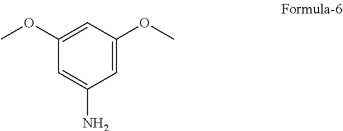
in presence of Cesium carbonate as a base, Palladium(II)acetate as a catalyst, Xantphos as a ligand in a suitable solvent to provide N-(3,5-dimethoxy phenyl)-3-(1-methylpyrazol-4-yl)quinoxaline-6-amine (Formula-7), optionally purifying the obtained compound with a suitable solvent, 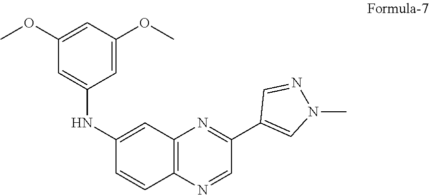
d) reacting the compound of formula-7 with N-(2-chloroethyl)-2-propanamine·HCl (Formula-8), 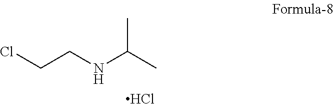
in presence of potassium hydroxide as a base, tetrabutylammonium bromide (TBAB) as a catalyst in a suitable solvent to provide N-(3,5-dimethoxyphenyl)-N′-(1-methylethyl)-N-[3-(1-methyl-1H-pyrazol-4-yl) quinoxalin-6-yl]ethane-1,2-diamine crude (Formula-1), followed by treating the obtained compound with IPA·HCl to provide Erdafitinib HCl salt compound of Formula-1a, optionally purifying the obtained compound with a suitable solvent, 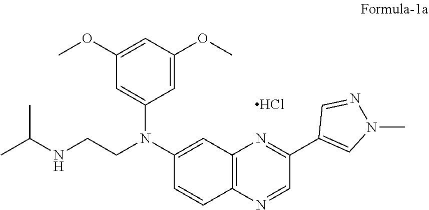 [SMILES: COc1cc(OC)cc(N(CCNC(C)C)c2ccc3ncc(-c4cnn(C)c4)nc3c2)c1 ; Cl]
e) treating the compound of formula-1a with sodium hydroxide in a suitable solvent to provide Erdafitinib compound of formula-1.

2. The process as claimed in claim-1, wherein,
In step-a, b, c, d & e) the suitable solvent is selected from alcoholic solvents, polar-aprotic solvents, hydrocarbon solvents, ester solvents, ether solvents, ketone solvents, chloro solvents, nitrile solvents and polar solvents such as water or mixtures thereof.

3. The process as claimed in claim-1, comprising of:
a) Treating 7-Bromo-1H-quinoxalin-2-one of formula-2 with thionyl chloride in presence of N, N-dimethylformamide as a catalyst in absence of a solvent to provide 7-Bromo-2-chloroquinoxaline (Formula-3), purifying the obtained compound with 15% ethyl acetate in hexane to obtain compound of formula-3 with high purity,
b) reacting the compound of formula-3 with 1-Methylpyrazole-4-boronic acid pinacol ester (Formula-4) in presence of sodium carbonate as a base, tetrakis(triphenyl phosphine)palladium [Pd(PPh₃)₄] as catalyst and triphenyl phosphine as ligand in dimethyl sulfoxide (DMSO) solvent to provide 7-Bromo-2-(1-methyl-1H-pyrazol-4yl)quinoxaline (Formula-5), purifying the obtained compound with 20% ethyl acetate in hexane to obtain compound of formula-5 with high purity,
c) reacting the compound of formula-5 with 3,5-Dimethoxyaniline of formula-6 in presence of Cesium carbonate as a base, Palladium(II)acetate as a catalyst, Xantphos as a ligand in 1,2-dimethoxyethane solvent to provide N-(3,5-dimethoxyphenyl)-3-(1-methylpyrazol-4-yl)quinoxaline-6-amine (Formula-7), purifying the obtained compound with 25% tetrahydrofuran in ethyl acetate solvent to obtain compound of formula-7 with high purity,
d) reacting the compound of formula-7 with N-(2-chloroethyl)-2-propanamine·HCl of formula-8 in presence of potassium hydroxide as a base, tetrabutylammonium bromide (TBAB) as a catalyst in aqueous tetrahydrofuran solvent to provide N-(3,5-dimethoxyphenyl)-N′-(1-methylethyl)-N-[3-(1-methyl-1H-pyrazol-4-yl)quinoxalin-6-yl]ethane-1,2-diamine crude (Formula-1), followed by treating the obtained compound with IPA·HCl in isopropyl alcohol solvent to provide Erdafitinib HCl salt compound of Formula-1a which was purified with mixture of acetone and dichloromethane to obtain compound of formula-1a with high purity,
e) treating the compound of formula-1a with sodium hydroxide in ethyl acetate to provide Erdafitinib compound of formula-1, purifying the obtained compound with aqueous acetone to provide pure Erdafitinib compound of formula-I.

4. The process as claimed in claim-1, wherein the N-(3,5-dimethoxyphenyl)-3-(1-methylpyrazol-4-yl)quinoxaline-6-amine (Formula-7) 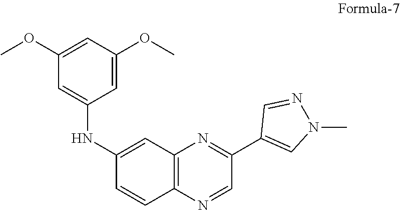
is prepared by reacting the compound of formula-5 with 3,5-Dimethoxyaniline of formula-6 in presence of Cesium carbonate as a base, Palladium(II)acetate as a catalyst, Xantphos as a ligand in 1,2-dimethoxyethane solvent.

5. A novel crystalline form-N of Erdafitinib HCl salt compound of formula-1a, which is characterized by:
i) Its powder X-ray diffractogram having peaks at about 6.2, 8.7, 13.0, 14.6, 21.4, 21.8 and 26.8±0.2 degrees 2-theta.
ii) Its powder X-ray diffraction pattern as shown in FIG. 1.

6. A process for the preparation of crystalline Form-N of Erdafitinib HCl compound of formula-1a as claimed in claim-5, comprising of:
a) Adding isopropyl alcohol to N-(3,5-dimethoxyphenyl)-N′-(1-methylethyl)-N-[3-(1-methyl-1H-pyrazol-4-yl)quinoxalin-6-yl]ethane-1,2-diamine (Formula-1 crude),
b) heating and stirring the reaction mixture,
c) adding IPA·HCl to the reaction mass,
d) cooling and stirring the reaction mixture,
e) adding a suitable solvent to the reaction mass,
f) filtering, washing and drying the compound to get the crystalline form-N of Erdafitinib HCl salt compound (Formula-1a).

7. The process as claimed in claim-6, comprising of:
a) Adding isopropyl alcohol to N-(3,5-dimethoxyphenyl)-N′-(1-methylethyl)-N-[3-(1-methyl-1H-pyrazol-4-yl)quinoxalin-6-yl]ethane-1,2-diamine (Formula-1 crude),
b) heating and stirring the reaction mixture,
c) adding IPA·HCl to the reaction mass,
d) cooling and stirring the reaction mixture,
e) adding acetone & dichloromethane to the reaction mass,
f) filtering, washing with acetone and drying the compound to get the crystalline form-N of Erdafitinib HCl (Formula-1a).

8. A process for the preparation of crystalline Form-A of Erdafitinib compound of formula-1, comprising of:
a) Adding a suitable solvent to Erdafitinib HCl compound (Formula-1a),
b) neutralizing the reaction mass with a suitable base,
c) adding a suitable solvent and heating the reaction mass,
d) stirring and cooling the reaction mixture,
e) filtering, washing and drying the compound to get the crystalline Form-A of Erdafitinib compound of formula-1.

9. The process as claimed in claim-8, wherein,
In step-b), the suitable base is sodium hydroxide.
In step-a) & c), the suitable solvents are selected from ether solvents, chloro solvents, ester solvents, alcohol solvents, ketone solvents, polar aprotic solvents, hydrocarbon solvents, nitrile solvents and polar solvents such as water or mixtures thereof.

10. The process as claimed in claim-8, comprising of:
a) Adding ethyl acetate to Erdafitinib HCl compound (Formula-1a),
b) neutralizing the reaction mass with sodium hydroxide,
c) adding aqueous acetone and heating the reaction mass,
d) stirring and cooling the reaction mixture,
e) filtering, washing and drying the compound to get the crystalline Form-A of Erdafitinib compound of formula-1.
